(19) United States
(12) Patent Application Publication
(10) Pub. No.: US 2022/0281800 A1
(43) Pub. Date: Sep. 8, 2022

(54) PURIFICATION OF AROMA CHEMICALS

(71) Applicant: BASF SE, Ludwigshafen am Rein (DE)

(72) Inventors: Eva Katharina HACKEMANN, Ludwigshafen am Rein (DE); Stephanie RENZ, Ludwigshafen am Rein (DE); Stephan MAURER, Ludwigshafen am Rein (DE); Volker KICKMANN, Ludwigshafen am Rein (DE); Markus JEGELKA, Ludwigshafen am Rein (DE); Manuel DANZ, Ludwigshafen am Rein (DE)

(21) Appl. No.: 17/631,898

(22) PCT Filed: Jul. 28, 2020
(86) PCT No.: PCT/EP2020/071204

§ 371 Date: Feb. 1, 2022

(30) Foreign Application Priority Data
Aug. 2, 2019 (EP) 19189760.2

(51) Int. Cl. C07C 68/08; C07C 68/02

(52) U.S. Cl. CPC C07C 68/08; C07C 68/02

(57) ABSTRACT

The presently claimed invention relates to a process for purification of aroma compounds by distillation. Specifically, it relates to a process for purification of carbonic esters of formula (I) using a combination of distillative processes.

The presently claimed invention relates to a process for purification of aroma compounds by distillation. Specifically, it relates to a process for purification of carbonic esters of formula (I) using a combination of distillative processes.

### BACKGROUND

Distillative processes are commonly used in chemical process technology to thermally separate mixtures of compounds of different relative volatilities and/or to thermally separate mutually soluble compounds.

Various process variants may be used for the continuous distillative separation of multi-component mixtures.

In the simplest case, a feed mixture composed of a low boiling fraction and a high boiling fraction is separated into its two fractions, i.e. a low boiling top fraction and a high boiling bottom fraction. In this case, the mixture to be separated is introduced in between the bottom and the top of the distillation column. The feed inlet divides the column into a rectifying section and a stripping section. The high boiling fraction is removed from the column in the bottom region. Part of the concentrate is evaporated using a heating unit (e.g. a natural circulation evaporator) installed in the bottom region. The low boiling fraction rises up inside the column as vapor, is withdrawn from the top of the column, and is condensed in a condenser.

Carbonic acid esters are valuable compounds for the preparation of tooth cleaning agents, mouthwashes, dental rinses, foodstuffs, drinks and cosmetics.

Carbonic acid esters are prepared via the corresponding chloroformates. The chloroformates are in turn obtained from the corresponding alcohols and phosgene. However, certain amounts of impurities are produced by this reaction, especially through chlorination of the respective alcohols, and these impurities must be removed by methods which may unfavourably affect the general economy of the process, wherein the chloroformates are used. Thus, in addition to the disadvantages described above, the presence of such impurities in chloroformates may result in generation of further impurities and/or by-products during the reaction of the chloroformates with alcohols to form carbonic acid esters and may require extensive purification of the desired carbonic acid esters.

The prior art discloses the purification of the carbonic acid esters using a thin film evaporation at low pressure with a yield of 70-80%. Further, the removal of impurities formed during the process of synthesis of carbonic acid esters was described to be difficult.

Consequently, there is a need to provide a purification method for carbonic acid esters which improves the yields of the desired carbonic acid esters while minimizing the amount of impurities and solvents within permissible limits and ensuring that the temperature sensitive carbonic acid esters are not degraded in the purification process.

### SUMMARY OF THE INVENTION

It was surprisingly found that the purification of carbonic acid esters by a combination of steam stripping and short path evaporation allows for the provision of carbonic acid esters with a high yield and a minimum amount of impurities or even no impurities at all.

Hence, in one aspect, the presently claimed invention relates to a method for the purification of a mixture comprising a carbonic esters of formula (I),

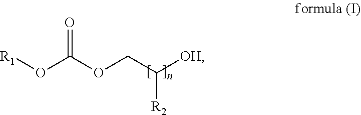

wherein

R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;

R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3;

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

comprising at least the steps of:

a) subjecting the mixture to steam stripping to obtain a stripped mixture; and

b) distillation of the stripped mixture of step a) by short-path evaporation to obtain the purified carbonic esters of formula (I).

In another aspect, the presently claimed invention relates to a method for the purification of a mixture comprising a compound of formula (I), wherein the compound of formula (I) is a compound of formula (IA),

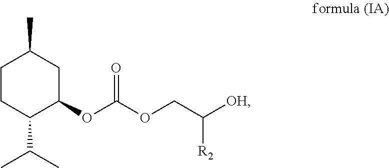

wherein R₂ is hydrogen or methyl.

### BRIEF DESCRIPTION OF THE FIGURES

FIG. 1—Flow chart of the purification process

### DETAILED DESCRIPTION OF THE INVENTION

Although the presently claimed invention will be described with respect to particular embodiments, this description is not to be construed in a limiting sense.

Before describing in detail exemplary embodiments of the presently claimed invention, definitions important for understanding the presently claimed invention are given. As used in this specification and in the appended claims, the singular forms of “a” and “an” also include the respective plurals unless the context clearly dictates otherwise. In the context of the presently claimed invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the presently claimed invention the term “consisting of” is considered to be a preferred embodiment of the term “comprising of”. If hereinafter a group is defined to comprise at least a certain number of embodiments, this is meant to also encompass a group which preferably consists of these embodiments only.

In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only and is not intended to limit the scope of the presently claimed invention that will be limited only by the appended claims. Unless defined otherwise, all technical and scientific terms used herein have the same meanings as commonly understood by one of ordinary skill in the art.

Unless otherwise indicated, the following definitions are set forth to illustrate and define the meaning and scope of the various terms used to describe the invention herein and the appended claims. These definitions should not be interpreted in the literal sense as they are not intended to be general definitions and are relevant only for this application.

It will be understood that “substitution”, “substituted” or “substituted with” means that one or more hydrogens of the specified moiety are replaced with a suitable substituent and includes the implicit proviso that such substitutions are in accordance with the permitted valence of the substituted atom and the substituent and results in a stable compound.

When any variable (for instance, R₁, R₂, R₃, R₄, R₅ etc.) or substituent has more than one occurrence, its definition on each occurrence is independent at every other occurrence. Also, combinations of substituents and/or variables are permissible, only if such combinations result in stable compounds.

The term “independently”, when used in the context of selection of substituents for a variable, it means that where more than one substituent is selected from many possible substituents, those substituents may be the same or different.

Salts of the compounds according to the invention can be formed in a customary manner, for example, by reacting the compound with an acid of the anion in question if the compounds according to the invention have a basic functionality or by reacting acidic compounds according to the invention with a suitable base.

The organic moieties or groups mentioned in the above definitions of the variables are like the term halogen—collective terms for individual listings of the individual group members.

The term “Cv-Cw” indicates the number of carbon atom possible in each case.

The term “C₁-C₁₀-alkyl” refers to a linear or branched saturated hydrocarbon group having 1 to 10 carbon atoms, for example, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “C₁-C₆-alkyl” refers to a linear or branched saturated hydrocarbon group having 1 to 6 carbon atoms, for example, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl propyl.

The term “C₃-C₁₀-alkenyl” refers to a linear or branched unsaturated hydrocarbon radical having 2 to 10 carbon atoms and a double bond in any position. Examples are “C₂-C₄-alkenyl” groups, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₃-C₁₀-alkynyl” refers to a linear or branched unsaturated hydrocarbon radical having 2 to 10 carbon atoms and containing at least one triple bond. Examples are “C₂-C₄ alkynyl” groups, such as ethynyl, prop-1-ynyl, prop-2-ynyl, but-1-ynyl, but-2-ynyl, but-3-ynyl, 1-methyl-prop-2-ynyl.

The term “C₅-C₁₀-cycloalkyl” refers to monocyclic saturated hydrocarbon radicals having 5 to 10 carbon ring members, such as cyclopropyl, cyclobutyl, cyclopentyl, cyclohexyl, cycloheptyl or cyclooctyl.

The term “C₅-C₁₀-cycloalkenyl” refers to monocyclic unsaturated hydrocarbon radical having 5 to 10 carbon ring members and a double bond in any position, for example cyclobutenyl, cyclopentenyl, cyclohexenyl or cyclooctenyl.

The term “substituted”, if not specified otherwise, refers to substituted with 1, 2 or maximum possible number of substituents. If substituents are more than one, then they are independently from each other are same or different, if not mentioned otherwise.

Meaning of the terms that are not defined herein are generally known to a person skilled in the art or in the literature.

In an embodiment, the presently claimed invention provides for a method for the purification of a mixture comprising a carbonic esters of formula (I),

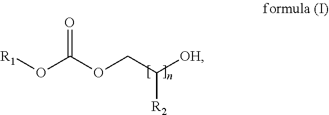

wherein

R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;

R₂ is selected from the group consisting of hydrogen, unsubstituted or substituted, linear or branched C₁-C₁₀-alkyl, unsubstituted or substituted, linear or branched C₂-C₁₀-alkenyl, un-substituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3;

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

comprising at least the steps of:

a) subjecting the mixture to steam stripping to obtain a stripped mixture; and

b) distillation of the stripped mixture of step a) by short-path evaporation to obtain the purified carbonic esters of formula (I).

### Synthesis of the Compound of Formula (I)

In one embodiment, the carbonic acid ester of formula (I) and its stereoisomers,

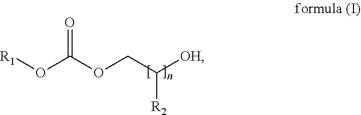

are prepared by a process

comprising at least the steps of:

A) reacting a compound of formula (II),

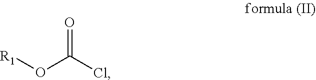

wherein

R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;

with an imidazole of formula (III),

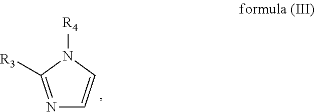

wherein

R₃ is hydrogen or unsubstituted, linear or branched, C₁-C₆-alkyl and R₄ is unsubstituted, linear or branched, C₁-C₆-alkyl;

to obtain a compound of formula (IV),

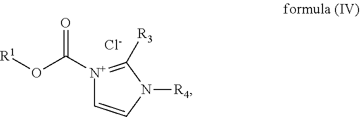

wherein

R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;

R₃ is hydrogen or unsubstituted, linear or branched C₁-C₆-alkyl; and

R₄ is unsubstituted, linear or branched C₁-C₆-alkyl;

and

B) reacting the compound of formula (IV) with a compound of formula (V),

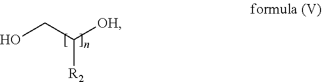

wherein

R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3; and

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

to obtain a compound of formula (I) and its stereoisomers.

In another embodiment, the presently claimed invention provides a process, wherein R₁ is selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl which are each unsubstituted or substituted by 1, 2 or 3 substituents selected from the group consisting of oxo, —F, —NO₂, —CN, —CF₃, —C(═O)CH₃, —C(═O)OCH₃, —NH—C(═O)(CH₃), —CH₂-phenyl, -phenyl; and

cyclopropyl, cyclobutyl, cyclopentyl, cyclohexyl, cyclohexenyl and cyclooctyl which are each unsubstituted or substituted by 1, 2, 3, or 4 substituents selected from the group consisting of methyl, ethyl, 1-propyl, 1-butyl, 1-pentyl, 1-hexyl, isopropyl, isopropenyl, isobutyl, tertiary butyl, isopentyl, 2-methylbutyl, 3-methylbutyl, -methoxy, -ethoxy, —F, —NO₂, —CN, —CF₃, —C(═O)CH₃, —C(═O)OCH₃, —NH—C(═O)CH₃.

More preferably, R₁ is cyclohexyl which is substituted by 1 or 2 substituents selected from the group consisting of methyl, ethyl, 1-propyl, isopropyl, isopropenyl and isobutyl.

Most preferably, R₁ is cyclohexyl which is substituted by methyl and isopropyl.

In yet another embodiment, R₂ is hydrogen or selected from group consisting of methyl, ethyl, 1-propyl, 1-butyl, 1-pentyl, 1-hexyl, isopropyl, isobutyl, tertiary butyl, isopentyl, 2-methylbutyl, 3-methylbutyl, cyclopropyl, cyclobutyl, cyclopentyl, cyclohexyl, cyclohexenyl and cyclooctyl which are each unsubstituted.

In another embodiment, n is 1, 2 or 3. Preferably, n is 1.

Preferably, when n is 2 or 3, the R₂, independently, is hydrogen or selected from the group consisting of methyl, ethyl, 1-propyl, 1-butyl, 1-pentyl, 1-hexyl, isopropyl, isobutyl, tertiary butyl, isopentyl, 2-methylbutyl, 3-methylbutyl, cyclopropyl, cyclobutyl, cyclopentyl, cyclohexyl and cyclooctyl, which are each unsubstituted.

In yet another embodiment, R₃ is selected from group consisting of hydrogen, methyl, ethyl, 1-propyl, 1-butyl, 1-pentyl, 1-hexyl, isopropyl, isobutyl, tertiary butyl, isopentyl, 2-methylbutyl and 3-methylbutyl. Preferably, R₃ is hydrogen or methyl.

In yet another embodiment, R₄ is selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl. In the most preferred embodiment, R₄ is methyl.

In another embodiment, the imidazole of formula (III) is selected from the group consisting of 1-methyl imidazole, 1-ethyl imidazole, 1-propyl imidazole, 1-isopropyl imidazole, 1-butyl imidazole, and 1,2-dimethyl imidazole.

In a preferred embodiment, the imidazole of formula (III) is 1,2-dimethyl imidazole or 1-methyl-imidazole.

In yet another embodiment, in step A) the molar ratio of the imidazole of formula (III) to the compound of formula (II) is in the range of ≥0.05:1.0 to ≤3.0:1.0 or preferably in the range of ≥0.06:1.0 to ≤2.75:1.0 or ≥0.075:1.0 to ≤2.5:1.0 or ≥0.25:1.0 to ≤2.5:1.0 or ≥0.5:1.0 to ≤2.5:1.0; more preferably in the range of ≥0.75:1.0 to ≤2.5:1.0 or ≥0.75:1.0 to ≤2.0:1.0 or ≥1.0:1.0 to ≤2.0:1.0.

In yet another embodiment, the at least step A) and step B) are carried out simultaneously.

In yet another embodiment, the at least step A) and step B) are carried out simultaneously, then as a base selected from group consisting of triethylamine, tripropylamine, tributylamine and N,N-diisopropyl-ethylamine can be used. In yet another embodiment the molar ratio of the base and the compound of formula (II) is in the range of ≥1.0: 1.0 to ≤3.0: 1.0, more preferably 2.0:1.0.

In yet another embodiment, in step A) the temperature is in the range of ≥10° C. to ≤80° C.; preferably the temperature is in the range of ≥15° C. to ≤75° C. or ≥15° C. to ≤70° C. or more preferably in the range of ≥15° C. to ≤65° C. or ≥15° C. to ≤60° C. or even more preferably in the range of ≥15° C. to ≤55° C. or ≥20° C. to ≤60° C. or ≥20° C. to ≤55° C.

In another embodiment, the at least one of the step A) and step B) is carried out in the presence of at least one non-polar solvent. The at least one compound of formula (III) and formula (IV) is dissolved or suspended in at least one non-polar solvent. Preferably the at least one non-polar solvent has dielectric constant in the range of ≥1.5 to ≤6.0 or in the range of ≥1.5 to ≤5.0 or even more preferably in the range of ≥1.5 to ≤4.5.

In a preferred embodiment, the at least one non-polar organic solvent is selected from the group consisting of aliphatic hydrocarbons, aromatic hydrocarbons and ethers.

In yet another preferred embodiment, the suitable aliphatic hydrocarbon is selected from the group consisting of pentane, hexane, heptane, cyclohexane and petroleum ether.

Further, in yet another preferred embodiment, a suitable aromatic hydrocarbon is selected from the group consisting of benzene, toluene and xylene.

In yet another preferred embodiment, the suitable ether solvent is selected from the group consisting of diethyl ether, diisopropyl ether, diethylene glycol dimethyl ether and methyl tert-butyl ether.

More preferably, the at least one non-polar solvent is selected from the group consisting of toluene, xylene, cyclohexane, heptane and methyl tert-butyl ether.

In another embodiment, in step B) the molar ratio of the compound of formula (II) to the compound of formula (V) is in the range of ≥1.0:2.0 to ≤1.0:20.0.

In yet another embodiment, tin step B) the temperature is in the range of ≥10° C. to ≤80° C.; preferably the temperature is in the range of ≥15° C. to ≤75° C. or ≥15° C. to ≤70° C. or more preferably in the range of ≥15° C. to ≤65° C. or ≥15° C. to ≤60° C. or even more preferably in the range of ≥15° C. to ≤55° C. or ≥20° C. to ≤60° C. or ≥20° C. to ≤55° C.

In another embodiment, there may be time intervals of seconds, minutes, hours or days between at least step A) and step B).

In yet another embodiment, the at least the compound of formula (IV) is isolated from the at least one non-polar solvent.

In one embodiment, the compound of general formula (II) is obtained by reacting a compound of formula (II′) with phosgene,

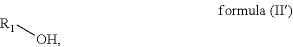

wherein

R is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl.

Preferably, R₁ is cyclohexyl or cyclohexenyl which is unsubstituted or substituted by 1, 2 or 3 substituents selected from the group consisting of oxo, methyl, ethyl, 1-propyl, 1-butyl, 1-pentyl, 1-hexyl, isopropyl, isopropenyl, isobutyl, tertiary butyl, isopentyl, 2-methylbutyl and 3-methylbutyl.

When R₁ is cyclohexyl which is substituted by methyl and isopropyl, the at least one compound of formula (IIA),

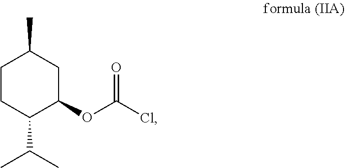

is obtained.

It has been observed that formula (IIA″), menthylchloride, is a potential impurity during the formation of menthylchloroformate (IIA). Also, it has been observed that the amount of formula (IIA″) increases when compound of formula (IIA) is stored for prolonged time or exposed to excessive heat owing to decomposition of compound of formula (IIA).

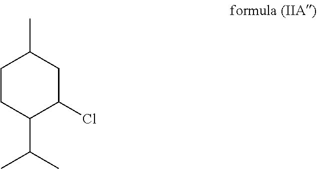

In one embodiment, the process for preparation of a compound of formula (IIA) involves removing a compound of formula (IIA″) from a compound of formula (IIA) comprising at least the steps of:

A) reacting the mixture comprising compound of formula (IIA) and compound of formula (IIA″) in at least one non-polar solvent

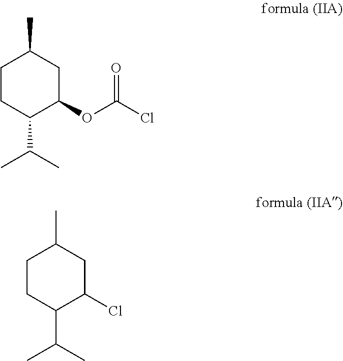

with an imidazole of formula (III),

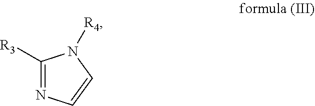

wherein R₃ is hydrogen or unsubstituted, linear or branched C₁-C₆-alkyl and R₄ is unsubstituted, linear or branched C₁-C₆-alkyl;

to obtain a mixture containing a compound of formula (IVA);

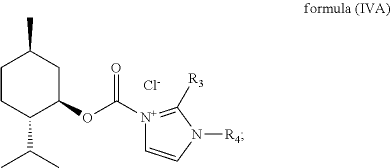

wherein R₃ is hydrogen or unsubstituted, linear or branched C₁-C₆-alkyl and R₄ is unsubstituted, linear or branched C₁-C₆-alkyl; and

B) optionally, isolating the compound of formula (IVA) from the mixture of step A).

In yet another embodiment, the isolated compound of formula (IVA) can be washed with at least one non-polar solvent. The compound of formula (IVA), so obtained, is free of compound of formula (IIA″). In yet another embodiment, the one non-polar solvent is selected from pentane, hexane, heptane, cyclohexane, petroleum ether, benzene, toluene xylene, diethyl ether, diisopropyl ether, diethylene glycol dimethyl ether and methyl tert-butyl ether.

In another embodiment, the isolated compound of formula (IVA) is reacted with compound of formula (V) in the presence of at least one non-polar solvent and 1-10 mol % imidazole of formula (III).

In yet another embodiment, step A) can be carried out in the presence of compound of formula (V).

In another embodiment, the compound of formula (V) is ethylene glycol or propylene glycol.

In one embodiment, the compound of formula (IA),

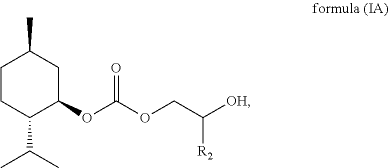

wherein R₂ is hydrogen or methyl;

whereby if R₂ is methyl, the formula (IA) comprises

the compound of formula (Ia)

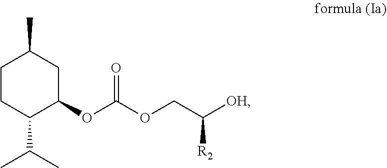

the compound of the formula (Ib)

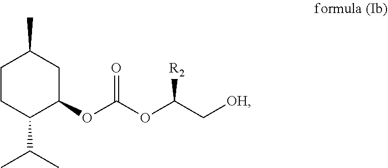

the compound of formula (Ic)

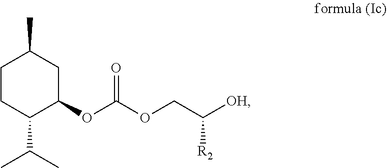

the compound of formula (Id)

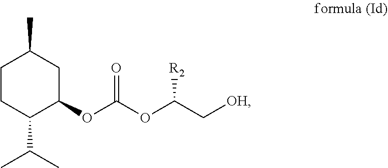

and its stereoisomers.

In yet another embodiment, the presently claimed invention provides the process, wherein at least the said compound of formula (I) and formula (IA), respectively, is

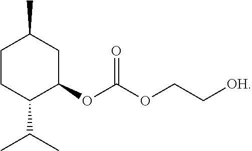 [SMILES: CC(C)[C@@H]1CC[C@@H](C)C[C@H]1OC(=O)OCCO]

In yet another embodiment, the presently claimed invention provides the process, wherein at least the said compound of formula (I) and formula (IA), respectively, is

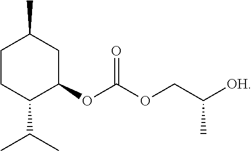 [SMILES: CC(C)[C@@H]1CC[C@@H](C)C[C@H]1OC(=O)OC[C@@H](C)O]

In yet another embodiment, the presently claimed invention provides the process, wherein at least the said compound of formula (I) and formula (IA), respectively, is

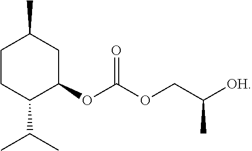 [SMILES: CC(C)[C@@H]1CC[C@@H](C)C[C@H]1OC(=O)OC[C@H](C)O]

### Purification of the Compound of Formula (I)

In an embodiment of the presently claimed invention, the purification of a mixture comprising a carbonic ester of formula (I) comprises at least the steps of:

a) subjecting the mixture to steam stripping to obtain a stripped mixture; and

b) distillation of the stripped mixture of step a) by short-path evaporation to obtain the purified carbonic esters of formula (I).

In an embodiment, the presently claimed invention provides a method for the purification of the carbonic ester of formula (I), wherein the mixture comprising the carbonic ester of formula (I) is subjected to steam stripping to separate at least one compound having a vapor pressure in the range of ≥0.0001 bar to ≤0.20 bar at 60° C.

In an embodiment, the presently claimed invention provides a method for the purification of the carbonic ester of formula (I), wherein the mixture comprising the carbonic ester of formula (I) is subjected to steam stripping to separate at least one compound having a vapor pressure in the range of ≥0.0001 bar to ≤0.20 bar at 60° C. as head product.

In an embodiment of the presently claimed invention, the steam stripping process separates at least one compound having a vapor pressure in the range of ≥0.0001 bar to ≤0.20 bar at 60° C., which is selected from the group consisting of non-polar organic solvents and impurities formed during the synthesis of the carbonic esters of formula (I) as head product.

In an embodiment of the presently claimed invention, the at least one compound having a vapor pressure in the range of ≥0.0001 bar to ≤0.20 bar at 60° C. is a non-polar solvent selected from the group consisting of aliphatic hydrocarbons like pentane, hexane, heptane, cyclohexane and petroleum ether, aromatic hydrocarbon like benzene, toluene and xylene, ethers like diethyl ether, diisopropyl ether, diethylene glycol dimethyl ether and methyl tert-butyl ether.

In another embodiment of the presently claimed invention, the at least one compound having a vapor pressure in the range of ≥0.0001 bar to ≤0.20 bar at 60° C. is an impurity formed during the synthesis of carbonic esters of formula (I) which are menthyl chloride and menthol.

In an embodiment of the presently claimed invention, the steam stripping is carried out in a column having a sump temperature in the range of ≥50° C. to ≤120° C. and head temperature in the range of ≥40° C. to ≤60° C. , more preferably having a sump temperature in the range of ≥80° C. to ≤120° C. and head temperature in the range of ≥45° C. to ≤60° C.

In an embodiment of the presently claimed invention, the steam stripping is carried out at a pressure in the range of ≥100 mbar to ≤200 mbar, more preferably in the range of ≥120 to ≤180 mbar.

In an embodiment of the presently claimed invention, the head product of the steam stripping process is further separated by batch distillation.

In another embodiment of the presently claimed invention, the batch distillation is carried out at a sump temperature range of ≥50° C. to ≤80° C. and head temperature in the range of ≥30° C. to ≤60° C., more preferably at a sump temperature range of ≥60° C. to ≤80° C. and head temperature in the range of ≥40° C. to ≤50° C.

In a further embodiment of the presently claimed invention, the batch distillation is carried out at a pressure in the range of ≥50 mbar to ≤150 mbar, more preferably in the range of ≥80 mbar to ≤120 mbar.

In an embodiment of the presently claimed invention, the distillation of the stripped mixture of step a) is subjected to a short-path evaporation to obtain the purified carbonic esters of formula (I).

In another embodiment of the presently claimed invention, the short path evaporation is carried out in the temperature range of ≥90° C. to ≤130° C., more preferably in the temperature range of ≥100° C. to ≤130° C.

In a further embodiment of the presently claimed invention, the short path evaporation is carried out at a pressure range of ≥0.10 mbar to ≤0.80 mbar, preferably in the range of ≥0.10 mbar to ≤0.60 mbar, more preferably in the range of ≥0.10 mbar to ≤0.50 mbar

In an embodiment of the presently claimed invention, the short path evaporation is carried out, wherein the area load of the stripped mixture is in the range of ≥1 to ≤100 kg/m² of evaporator area per hour, preferably in the range of ≥1 to ≤50 kg/m² of evaporator area per hour.

The term “short path evaporation” used in the method according to the presently claimed invention comprises the evaporation and subsequent condensation of corresponding compounds. For the purposes of this invention, “short path evaporation” is a thermal separation operation using a short path evaporator.

For the purposes of this invention, a short path evaporator is an evaporator in which “the condenser is integrated into the evaporator body so that the evaporated components only travel a very short distance in the vapour phase [ . . . ]”, cf. Fluidverfahrenstechnik: Grundlagen, Methodik, Technik, Praxis“, Ralf Goedecke, Publisher: John Wiley & Sons; 2011; page 643, Item 7.3.2.7.

For the purposes of this invention, the term “short path evaporation” also includes the so-called short path distillations.

In one embodiment, the short path evaporation is preferably performed in a corresponding short path evaporator with internal condenser and continuous mixing of the substance film to be separated on the evaporator surface.

The principle of short-path evaporation is based on the fact that a substance mixture fed to the evaporator is heated at an evaporator surface and the thereby evaporating components of the substance mixture condense at a condenser surface opposite the evaporator surface. In order to minimize pressure losses, the distance between the evaporator surface and the condenser surface is regularly chosen to be very small. The distance from the evaporator surface to the condenser (or condenser surface) is preferably a few centimetres.

A common short path evaporator preferred for the purposes of this invention comprises a cylindrical body with an external heating jacket and an internal wiper system so that evaporation can occur with continuous mixing of the substance film to be separated. Short path evaporators suitable for the purpose of this invention are commercially available, e.g. from UIC GmbH or Buss-SMS-Canzler GmbH.

In one embodiment, the Short path evaporator used in the process of the presently claimed invention usually comprises an evaporator surface and a condenser surface. In the context of this invention, the evaporator surface refers to the evaporator surface of the short path evaporator used and the condenser surface to the condenser surface of the short-path evaporator used.

In one embodiment, the above-mentioned temperature for performing the short path evaporation is the mean temperature of the heating medium used to heat the evaporator surface in the short path evaporator. The mean temperature of the heating medium is the arithmetic mean of the inlet and outlet temperatures of the heating medium.

In one embodiment, the evaporator surface and the condenser surface of the short path evaporator are directly opposed to each other when carrying out the procedure according to the presently claimed invention. The evaporator surface and the condenser surface of the short-path evaporator are arranged in a cylindrical manner, whereby two cylinders are placed one inside the other in the cylindrical arrangement.

In one embodiment, the pressure within the pressure range defined in the text above is lowered to such an extent that the mean free path of the evaporated particles in the vapor space is greater than the distance between the evaporator surface and the condenser surface (molecular distillation). The mean free path length can be determined according to known methods. The required pressure therefore depends, among other things, on the dimensions of the apparatus and the vapour pressure of the substance to be distilled at the selected temperature (cf. Kirk Othmer, Encyclopedia of chemical technology, 4th Ed., Wiley,

Vol. 8, page 349). Suitable arrangements are e.g. described in: H J L Burgess (ed), Molecular Stills, Chapman and Hall, 1963.

In one embodiment, an apparatus arrangement, comprising the evaporator surface and the condenser surface, can be designed in almost any geometrical form as long as a short path evaporation is possible. It is preferable that the two surfaces are directly opposite each other so that the molecules can pass unhindered from the evaporator surface to the condenser surface. For example, a plane-parallel arrangement of the two surfaces or a cylindrical arrangement in which two cylinders are placed one inside the other and the directly opposite surfaces of the two cylinders form the evaporator and condenser surfaces can be considered. The evaporator surface is heated in a suitable manner, generally by devices on the back, and the condenser surface is also cooled in a suitable manner, generally also by devices on the back.

In one embodiment, the stripped mixture to be distilled is fed into the upper end of the apparatus and distributed evenly over the inner circumference of the evaporator by the rotating wiper system. The product flows downwards as a film due to gravity on the externally heated evaporator surface. In order to ensure uniform wetting of the evaporator surface, intensive mixing and high turbulence in the product film and thus increase the evaporation performance, the well-known and common wiper systems can be used.

In an embodiment of the presently claimed invention, the purified carbonic ester of formula (I) has a solvent content of ≤30 ppm, preferably the solvent content of ≤20 ppm, more preferably of ≤10 ppm.

In an embodiment of the presently claimed invention, the purified carbonic ester of formula (I) has a menthyl chloride content of ≤200 ppm, preferably ≤150 ppm, more preferably ≤100 ppm

In an embodiment of the presently claimed invention, the process could be a batch process or a continuous process.

The presently claimed invention is associated with at least one of the following advantages:

1. Carbonic acid esters of formula (I) are obtained in a high yield with a very low content of toluene (i.e. <10 ppm) and methyl chloride (i.e. <100 ppm) by using two process steps only.

2. The solvents which are used for the preparation of the carbonic acid esters of formula (I) are easily recovered and recycled.

3. The process of the presently claimed invention can be used to purify the carbonic acid esters of formula (I) which are thermally sensitive. Hence, the carbonic acid esters of formula (I) do not degrade during the purification.

In the following, there is provided a list of embodiments to further illustrate the present disclosure without intending to limit the disclosure to the specific embodiments listed below.

### Embodiments

1. A method for the purification of a mixture comprising a carbonic esters of formula (I),

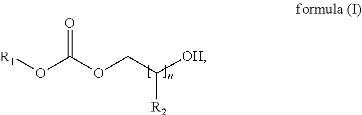

wherein

R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;

R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3;

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

comprising at least the steps of:

a) subjecting the mixture to steam stripping to obtain a stripped mixture; and

b) distillation of the stripped mixture of step a) by short-path evaporation to obtain the purified carbonic esters of formula (I).

2. The method according to embodiment 1, wherein the mixture is obtained by:

A) reacting a compound of formula (II),

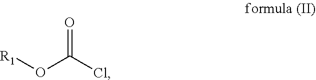

wherein R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₃-C₁₀-cycloalkyl and unsubstituted or substituted C₅-C₁₀-cycloalkenyl with an imidazole of formula (III),

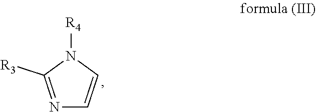

wherein R₃ is hydrogen or unsubstituted, linear or branched, C₁-C₆-alkyl and R₄ is unsubstituted, linear or branched, C₁-C₆-alkyl;

to obtain a compound of formula (IV),

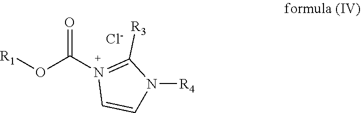

wherein R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; R₃ is hydrogen or unsubstituted, linear or branched C₁-C₆-alkyl; and

R₄ is unsubstituted, linear or branched C₁-C₆-alkyl; and

B) reacting the compound of formula (IV) with a compound of formula (V),

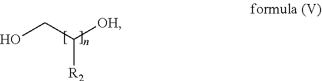

wherein R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3; and

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀cycloalkenyl;

to obtain a compound of formula (I).

3. The method according to embodiment 1, wherein the compound of formula (I) is a compound of formula (IA),

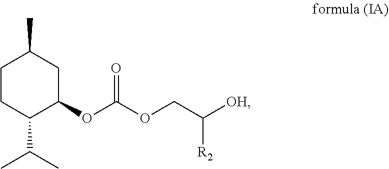

wherein R₂ is hydrogen or methyl.

4. The method according to embodiment 1, wherein in step a) the steam stripping is carried out in a stripping column having a sump temperature in the range of ≥50° C. to ≤120° C. and head temperature in the range of ≥40° C. to ≤60° C.

5. The method according to any of embodiments 1 to 4, wherein the steam stripping is carried out at a pressure in the range of ≥100 mbar to ≤200 mbar.

6. The method according to any of embodiments 1 to 5, wherein in step a) the mixture comprises at least one compound having a vapor pressure in the range of ≥0.0001 bar to ≤0.20 bar at 60° C.

7. The method according to embodiment 6, wherein the at least one compound having a vapor pressure in the range of ≥0.0001 bar to ≤0.20 bar at 60° C. is selected from the group consisting of non-polar organic solvents and impurities formed during the synthesis of the carbonic esters of formula (I).

8. The method according to embodiment 7, wherein the non-polar organic solvents are selected from the group consisting of aliphatic hydrocarbons, aromatic hydrocarbons and ethers.

9. The method according to embodiment 8, wherein the aliphatic hydrocarbons are selected from the group consisting of pentane, hexane, heptane, cyclohexane and petroleum ether.

10. The method according to embodiment 8, wherein the aromatic hydrocarbons are selected from the group consisting of benzene, toluene and xylene.

11. The method according to embodiment 8, wherein the ethers are selected from the group consisting of diethyl ether, diisopropyl ether, diethylene glycol dimethyl ether and methyl tert-butyl ether.

12. The method according to any of embodiments 7 to 11, wherein the non-polar organic solvents are selected from the group consisting of toluene, xylene, cyclohexane, heptane and methyl tert-butyl ether.

13. The method according to embodiment 7, wherein the impurities formed during the synthesis of carbonic esters of formula (I) are menthyl chloride and menthol.

14. The method according to embodiment 7, wherein the mixture comprising at least one compound having a vapor pressure in the range of ≥0.0001 bar to ≤0.20 bar at 60° C. is further separated by batch distillation.

15. The method according to embodiment 14, wherein the batch distillation is carried out at a sump temperature range of ≥50° C. to ≤80° C. and head temperature in the range of ≥30° C. to ≤60° C.

16. The method according to embodiment 14 or 15, wherein the batch distillation is carried out at a pressure in the range of ≥50 mbar to ≤150 mbar.

17. The method according to any of embodiments 1 to 16, wherein in step b) the temperature is in the range of ≥90° C. to ≤130° C.

18. The method according to any of embodiment 1 to 17, wherein in step b) the pressure is in the range of ≥0.10 mbar to ≤0.80 mbar.

19. The method according to any of embodiments 1 to 18, wherein in step b) the area load of the stripped mixture is in the range of ≥1 to ≤50 kg per m² of evaporator area per hour.

20. The method according to any of embodiments 1 to 19, wherein the method is a continuous method.

21. A method for the purification of a mixture comprising a carbonic esters of formula (I),

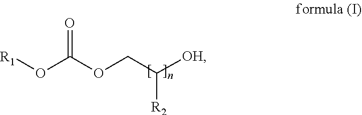

as defined in any of embodiments 1 to 20,

comprising at least the steps of:

a1) subjecting the mixture to steam stripping at a sump temperature in the range of ≥50° C. to ≤120° C. and head temperature in the range of ≥40° C. to ≤60° C. and a pressure in the range of ≥100 mbar to ≤200 mbar to obtain a stripped mixture and a mixture comprising at least one compound having a vapor pressure in the range of ≥0.0001 bar to ≤0.20 bar at 60° C.;

b1) batch distillation at a pressure in the range of ≥50 to ≤150 mbar of the mixture comprising at least one compound having a vapor pressure in the range of ≥0.0001 bar to ≤0.20 bar at 60° C.

c1) distillation of the stripped mixture of step a) by short path evaporation at a temperature in the range of ≥90° C. to ≤130° C. and a pressure in the range of ≥0.10 mbar to ≤0.80 mbar to obtain the purified carbonic esters of formula (I).

22. The method according to any of embodiments 1 to 21, wherein the purified carbonic ester of formula (I) has a solvent content of ≤30 ppm.

23. The method according to embodiment 22, wherein the purified carbonic ester of formula (I) has a solvent content of ≤10 ppm.

24. The method according to any of embodiments 1 to 21, wherein the carbonic ester of formula (I) has a menthyl chloride content of ≤200 ppm.

25. The method according to embodiment 24, wherein the carbonic ester of formula (I) has a menthyl chloride content of ≤100 ppm

While the presently claimed invention has been described in terms of its specific embodiments, certain modifications and equivalents will be apparent to those skilled in the art and are intended to be included within the scope of the presently claimed invention.

### EXAMPLES

The presently claimed invention is illustrated in detail by non-restrictive working examples which follow. More particularly, the test methods specified hereinafter are part of the general disclosure of the application and are not restricted to the specific working examples.

### I) Apparatus

Steam stripping column: 30 mm glass column packed with 1000 mm Montz A3 500 packing material

Short path evaporator: UIC laboratory short path evaporator KDL 5, 500 cm²

Batch distillation: 43 mm glass column packed with 13550 mm Montz A3 1000 packing material

### II) Preparation of menthyl propyleneglycol carbonate and menthyl ethyleneglycol carbonate

### a) Preparation of menthyl propylene glycol carbonate

In a first 1 L double-jacketed reactor with overhead stirrer, toluene (600 mL) and 1,2-dimethylimidazole (33 g, 0.34 mol) were placed at 25° C. Menthylchloroformate (75.1 g, purity approx. 96%, approx. 0.33 mol) which contained 2.5 w % menthylchloride (corresponding to 1.88 g) was added over 2 h at 25° C. After complete addition, stirring was continued for 30 min. The acyl-imidazolium-salt precipitated, and the resulting suspension was filtered and the solid was washed twice with toluene (3×300 mL). The mother liquor and the toluene of the two washing steps contained the menthylchloride (2.02 g). The acyl-imidazolium salt, essentially free of menthylchloride, was resuspended in toluene (300 ml).

In a second 1 L double-jacketed reactor, 1,2-propanediol (248.8 g, 3.27 mol) and 1,2-dimethylimidazole (1.1 g, 0.01 mol) were placed at 50° C. The suspension from the first reactor was then dosed into the second reactor over 90 min at 50° C. After complete addition, stirring was continued at 50° C. for 30 min. Then, the biphasic reaction mixture was cooled to 25° C. and the phases were separated. The glycol-phase was reextracted twice with toluene (2×60 mL) and the united toluene phases were washed with 5% aq. NaHCO₃-solution (300 mL) and water (2×300 mL). The solvent was removed using a thin-film evaporator (70° C., 180 mbar) and the product was obtained as a clear viscous liquid (76% yield).

The remaining menthylchloride content was 0.01%.

### b) Preparation of menthyl ethylene glycol carbonate

1,2-propanediol in example II a is replaced by ethylene glycol in the synthesis of menthyl ethyleneglycol carbonate.

### III) Purification of menthyl propyleneglycol carbonate MPC/menthyl ethyleneglycol carbonate MGC

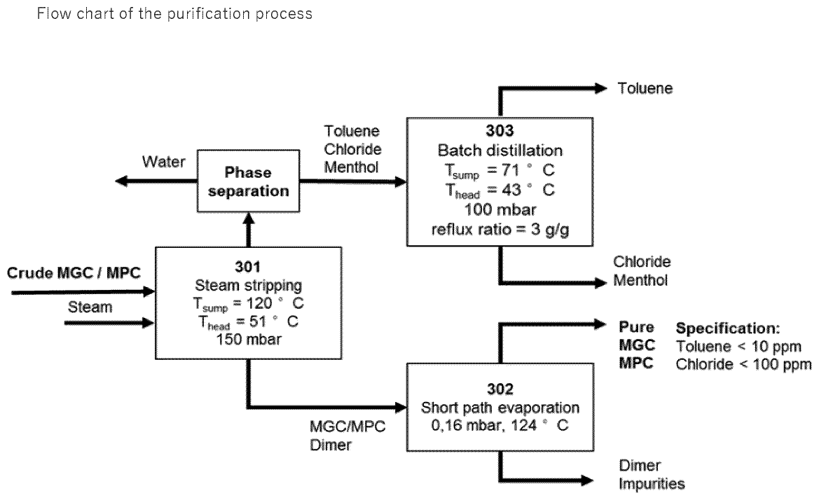

Example 1 and 2 describe the purification process for MPC and MGC. Reference is made to FIG. 1 for the steps in purification.

### Example 1—Purification of menthyl propyleneglycol carbonate

The crude MPC (having the following content, toluene 56.52 wt. %, menthol 1.06 wt. %, menthyl chloride 0.21 wt. %, MPC 40.37 wt. %, dimer 1.46 wt. %) was subjected to steam stripping (301) in a column, wherein the sump temperature did not exceed 120° C. and the pressure was in between 100 mbar and 200 mbar. Following steam stripping, MPC and impurities were separated as bottom product and toluene and menthyl chloride as the head product.

The head product was subjected to phase separation to separate the water phase and the toluene phase. The toluene phase was further distilled using a distillation column (303) at a sump temperature of 71° C. and head temperature of 43° C. and a pressure of 100 mbar to separate menthyl chloride and menthol.

MPC was distilled overhead to separate dimer and impurities using a short path evaporator (302). The pressure was in the range of 0.16-0.4 mbar and the temperature in between 119-124° C. The total yield of MPC was between 85-91%.

### Example 2—Purification of menthyl ethyleneglycol carbonate

The crude MGC (having the following content, toluene 62.89 wt. %, menthol 1.15 wt. %, menthyl chloride 0.26 wt. %, MGC 32.15 wt. %, dimer 4.77 wt. %) was subjected to steam stripping (301) in a column, wherein the sump temperature did not exceed 120° C. and the pressure was in between 100 mbar and 200 mbar. Following steam stripping, MGC and impurities were separated as bottom product and toluene and menthyl chloride as the head product.

The head product was subjected to phase separation to separate the water phase and the toluene phase. The toluene phase was further distilled using a distillation column (303) at a sump temperature of 71° C. and head temperature of 43° C. and a pressure of 100 mbar to separate menthyl chloride and menthol.

MGC was distilled overhead to separate dimer and impurities using a short path evaporator (302). The pressure was in the range of 0.16-0.4 mbar and temperature between 119-124° C. The total yield of MGC was between 85-91%.

### Optimization of Process Parameters

### Desired Specification of Impurities and Solvents

Solvent (toluene)<10 ppm

Menthyl chloride (MC)<100 ppm

L-Menthol<2 wt. %

Dimer<3 wt. %

### Example 3—Influence of menthyl chloride and menthol Concentration on the Purification of MGC in the Crude

To study the influence of menthyl chloride and menthol concentration on the purification of MGC, experiments were set up by adding menthyl chloride or menthol to the crude mixture such that in experiment no. 3a the menthyl chloride concentration was 1 wt. % and in experiment no. 3b to 3d the menthol concentration was 1 wt. %. Neither menthyl chloride nor L-menthol was added to experiment no. 3e to 3g. These crude products were then subjected to the purification process as per example 2.

Table 1 depicts the final concentration of all the specified components. AH the components are within the desired specification. However as illustrated in the table 1, the yield of the final product MGC is varying. The yield was improved by varying the temperature and the pressure in the short path evaporator.

TABLE 1
Influence of menthyl chloride and menthol concentration
on the purification of MGC in the crude.

| | Final concentration | | | | | |
|---|---|---|---|---|---|---|
| | | | | menthyl | | |
| | MGC | toluene | L-menthol | Chloride | dimer | |
| | Desired levels | | | | | Yield |
| Expt no. | >95 wt % | <10 ppm | <2 wt. % | <100 ppm | <3 wt. % | MGC (%) |
| 3a | 98.70 | <10 | 0.28 | 43 | 0.80 | 68.5 |
| 3b | 98.19 | <10 | 0.30 | 41 | 1.45 | 79.3 |
| 3c | 98.57 | <10 | 0.11 | 43 | 1.47 | 73.9 |
| 3d | 98.09 | <10 | 0.18 | 37 | 2.35 | 80.9 |
| 3e-3f | 97.27 | <10 | 0.21 | 41 | 3.03 | 87.3 |
| 3g | 97.72 | <10 | 0.19 | 41 | 2.49 | 90.3 |

### Example 4: Influence of Temperature and Pressure

MGC and MPC are temperature sensitive (onset temperature MGC: 130 -135° C., onset temperature MPC: 110-120° C.). If the onset temperature is exceeded, MGC/MPC reacts to menthol and ethylene carbonate. This results in an increase of these two side products and a decrease of the MGC/MPC yield.

An overview of the vapor pressures of the significant components at 60° C. are given in table 2.

In step 301 the temperature in the sump should be at maximum 120° C. At this conditions, toluene, MC and menthol are stripped out of the MGC using water steam.

In step 302 the chosen pressure should be lower than 1 mbar. The optimal conditions in the short path evaporator regarding yield and dimer specification are 0.4 mbar and 119° C. In step 303 the separation of MC and toluene is easy, due to a big vapor pressure difference

TABLE 2
Vapor pressure of various components

| | Vapor pressure (bar) | | | | | | |
|---|---|---|---|---|---|---|---|
| Temp | | | | | MPC- | menthyl | |
| ° C. | MPC | MGC | toluene | dimer 1 | Dimer 1 | chloride | menthol |
| 60 | 0.000102 | 9.3087E−06 | 0.18514 | 4.20E−08 | 2.74E−08 | 0.0048669 | 0.0016439 |

### Example 4A: Influence of Temperature and Pressure in Short Path Evaporation

Experiments 4a to 4g are on the similar lines as indicated for 3a to 3g except the variation in pressure and temperature as indicated in table 3.

TABLE 3
Influence of Temperature and Pressure for the recovery
of MGC in the short path evaporation step

| | Final concentration | | | | | | | |
|---|---|---|---|---|---|---|---|---|
| | | | | menthyl | | | | |
| | MGC | toluene | L-menthol | Chloride | dimer | | | |
| | Desired levels | | | | | yield | P | |
| Expt no. | >95 wt % | <10 ppm | <2 wt. % | <100 ppm | <3 wt. % | MGC (%) | (mbar) | T (° C.) |
| 4a | 98.70 | <10 | 0.28 | 43 | 0.80 | 68.5 | 0.43 | 119 |
| 4b | 98.19 | <10 | 0.30 | 41 | 1.45 | 79.3 | 0.28 | 118 |
| 4c | 98.57 | <10 | 0.11 | 43 | 1.47 | 73.9 | 0.33 | 121 |
| 4d | 98.09 | <10 | 0.18 | 37 | 2.35 | 80.9 | 0.21 | 123 |
| 4e-4f | 97.27 | <10 | 0.21 | 41 | 3.03 | 87.3 | 0.16 | 124 |
| 4g | 97.72 | <10 | 0.19 | 41 | 2.49 | 90.3 | 0.16 | 124 |

Experiments 5a to 5g are on the similar lines as indicated for 3a to 3g except the variation in pressure and temperature as indicated in table 4.

TABLE 4
Influence of Temperature and Pressure for the recovery
of MPC in the short path evaporation step

| | Final concentration | | | | | | | |
|---|---|---|---|---|---|---|---|---|
| | Desired levels | | | | | | | |
| | | | D-L- | | | | | |
| | MPC > | toluene < | menthol < | chloride < | dimer < | Yield | P | |
| Expt. No. | 95 wt. % | 10 ppm | 2 wt. % | 100 ppm | 3 wt. % | MPC [%] | (mbar) | T(° C.) |
| 5a | 98.06 | <10 | 0.17 | 61 | 1.34 | 77.2 | 0.4 | 118 |
| 5b | 98.41 | <10 | 0.33 | 57 | 0.63 | 92.5 | 0.4 | 118 |
| 5c | 98.32 | <10 | 0.26 | 58 | 0.72 | 91.2 | 0.4 | 118 |
| 5d | 98.40 | <10 | 0.32 | 46 | 0.69 | 92.2 | 0.4 | 118 |
| 5e-5f | 98.56 | <10 | 0.30 | 46 | 0.57 | 92.4 | 0.4 | 118 |
| 5g | 97.86 | <10 | 0.76 | 47 | 0.55 | 91.5 | 0.4 | 118 |

1.-18. (canceled)

19. A method for the purification of a mixture comprising a carbonic ester of formula (I), 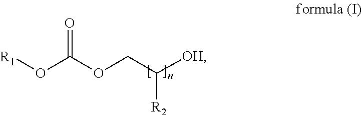
wherein
R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;
R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
n is 1, 2 or 3;
wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
comprising at least the steps of:
a) subjecting the mixture to steam stripping to obtain a stripped mixture; and
b) distillation of the stripped mixture of step a) by short-path evaporation to obtain the purified carbonic esters of formula (I).

20. The method according to claim 19, wherein the mixture is obtained by:
A) reacting a compound of formula (II), 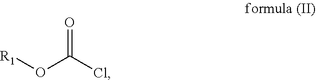
wherein is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl,
with an imidazole of formula (III), 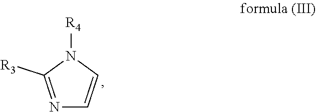
wherein R₃ is hydrogen or unsubstituted, linear or branched C₁-C₆-alkyl and R4 is unsubstituted, linear or branched C₁-C₆-alkyl;
to obtain a compound of formula (IV), 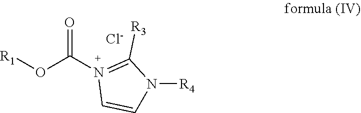
wherein is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;
R₃ is hydrogen or unsubstituted, linear or branched C₁-C₁₀-alkyl; and
R₄ is unsubstituted, linear or branched C₁-C₆-alkyl; and
B) reacting the compound of formula (IV) with a compound of formula (V), 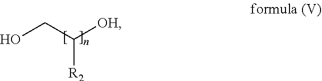
wherein R is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-unsubstituted or substituted C₅-C₁₀-cycloalkyl and unsubstituted or substituted C₅-C₁₀cycloalkenyl;
n is 1, 2 or 3; and
wherein when n is 2 or 3; R₂; independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl,
to obtain a compound of formula (I).

21. The method according to claim 19, wherein the compound of formula (I) is a compound of formula (IA), 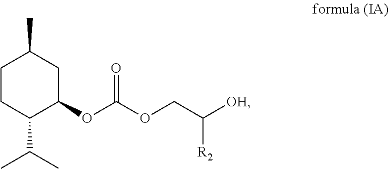
wherein R₂ is hydrogen or methyl.

22. The method according to claim 19, wherein in step a) the steam stripping is carried out in a stripping column having a sump temperature in the range of ≥50° C. to ≤120° C. and head temperature in the range of ≥40° C. to ≤60° C.

23. The method according to claim 19, wherein the steam stripping is carried out at a pressure in the range of ≥100 mbar to ≤200 mbar.

24. The method according to claim 19, wherein in step a) the mixture comprises at least one compound having a vapor pressure in the range of ≥0.0001 bar to ≤0.20 bar at 60° C.

25. The method according to claim 24, wherein the at least one compound having a vapor pressure in the range of ≥0.0001 bar to ≤0.20 bar at 60° C. is selected from the group consisting of non-polar organic solvents and impurities formed during the synthesis of the carbonic esters of formula (I).

26. The method according to claim 25, wherein the non-polar organic solvents are selected from the group consisting of aliphatic hydrocarbons, aromatic hydrocarbons and ethers.

27. The method according to claim 25, wherein the impurities formed during the synthesis of carbonic esters of formula (I) are menthyl chloride and menthol.

28. The method according to claim 25, wherein the mixture comprising at least one compound having a vapor pressure in the range of ≥0.0001 bar to ≤0.20 bar at 60° C. is further separated by batch distillation.

29. The method according to claim 28, wherein the batch distillation is carried out at a sump temperature range of ≥50° C. to ≤80° C. and head temperature in the range of ≥30° C. to ≤60° C.

30. The method according to claim 28, wherein the batch distillation is carried out at a pressure in the range of ≥50 mbar to ≤150 mbar.

31. The method according to claim 19, wherein in step b) the temperature is in the range of ≥90° C. to ≤130° C.

32. The method according to claim 19, wherein in step b) the pressure is in the range of ≥0.10 mbar to ≤0.80 mbar.

33. The method according to claim 19, wherein in step b) the area load of the stripped mixture is in the range of ≥1 to ≤50 kg per m² of evaporator area per hour.

34. A method for the purification of a mixture comprising a carbonic ester of formula (I), 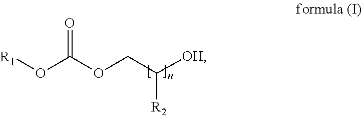
as defined in claim 19,
comprising at least the steps of:
a1) subjecting the mixture to steam stripping at a sump temperature of in the range of ≥50° C. to ≤120° C. and head temperature in the range of ≥40° C. to ≤60° C. and a pressure in the range of ≥100 mbar to ≤200 mbar to obtain a stripped mixture and a mixture comprising at least one compound having a vapor pressure in the range of ≥0.0001 bar to ≤0.20 bar at 60° C.;
b1) batch distillation at a pressure in the range of ≥50 to ≤150 mbar of the mixture comprising at least one compound having a vapor pressure in the range of ≥0.0001 bar to ≤0.20 bar at 60° C.
c1) distillation of the stripped mixture of step a) by short path evaporation at a temperature in the range of ≥90° C. to ≤130° C. and a pressure in the range of ≥0.10 mbar to ≤0.80 mbar to obtain the purified carbonic esters of formula (I).

35. The method according to claim 19, wherein the purified carbonic ester of formula (I) has a solvent content of ≤30 ppm.

36. The method according to claim 19, wherein the carbonic ester of formula (I) has a menthyl chloride content of ≤200 ppm.
